(19) United States
(12) Patent Application Publication
(10) Pub. No.: US 2017/0189345 A1
(43) Pub. Date: Jul. 6, 2017

(54) NANOENCAPSULATION OF ANTIGEN-BINDING MOLECULES

(71) Applicant: AbbVie Deutschland GmbH & Co. KG, Wiesbaden (DE)

(72) Inventors: Anamarija CURIC, Ludwigshafen (DE); Jan-Peter MÖSCHWITZER, Ludwigshafen (DE)

(21) Appl. No.: 15/315,293

(22) PCT Filed: May 29, 2015
(86) PCT No.: PCT/EP2015/061926

§ 371 Date: Nov. 30, 2016

Related U.S. Application Data
(60) Provisional application No. 62/005,163, filed on May 30, 2014.

(30) Foreign Application Priority Data
May 30, 2014 (EP) 14170537.6

(51) Int. Cl. A61K 9/51; C07K 16/44; C07K 16/22; A61K 39/395

(52) U.S. Cl. CPC A61K 9/5138; A61K 39/39591; A61K 39/395; C07K 16/44; C07K 16/22

(57) ABSTRACT

The present invention relates to nanospheres comprising a polymeric matrix and antigen-binding molecules esterase-releasably incorporated therein. The polymeric matrix is formed by poly(alkyl cyanoacrylates) and/or alkoxy derivatives thereof. The invention further relates to methods for preparing and compositions comprising such nanospheres.

The present invention relates to nanospheres comprising a polymeric matrix and antigen-binding molecules esterase-releasably incorporated therein. The invention further relates to methods for preparing and compositions comprising such nanospheres.

### BACKGROUND OF THE INVENTION

Nanoparticles have been studied as drug delivery systems and in particular as possible sustained release systems for targeting drugs to specific sites of action within the patient. The term “nanoparticles” is generally used to designate polymer-based particles having a diameter in the nanometer range. Nanoparticles include particles of different structure, such as nanospheres and nanocapsules. Nanoparticles based on biocompatible and biodegradable polymers such as poly(alkyl cyanoacrylates) have been studied over the past three decades and are of particular interest for biomedical applications (cf. Couvreur et al., J Pharm Pharmacol, 1979, 31:331-332; Vauthier et al., Adv. Drug Deliv. Rev. 2003, 55:519-548). They can be prepared by miniemulsion polymerization (cf., e.g., Reimold et al., Eur. J. Pharm. Biopharm. 2008, 70:627-632; Vauthier et al., Adv. Drug Deliv. Rev. 2003, 55:519-548) and their surface can be modified in different ways allowing the nanoparticles to accumulate in specific target organs or tissues (cf. Vauthier et al., Adv. Drug Deliv. Rev. 2003, 55:519-548). For example, the attachment of antibodies to the surface of nanoparticles has been described (cf., e.g., Hasadsri et al., J Bio Chem, 2009, 284:6972-6981). Moreover, nanoparticles coated with polysorbate 80 have been shown to transport drugs which are normally unable to cross the blood-brain barrier across this barrier (cf. WO 2007/088066; Kreuter et al., J. Drug Target. 2002, 10(4):317-325; Reimold et al., Eur. J. Pharm. Biopharm. 2008, 70:627-632).

Despite ample research in the field of nanoparticles, little is known about the encapsulation of antibodies by incorporation into the polymeric matrix of nanospheres. Antibodies are relatively large molecules (˜150 kDa for an IgG) with great therapeutic potential. Due to their size, antibodies are normally not able to cross biological barriers such as the blood-brain barrier. Moreover, proteins such as antibodies are potentially susceptible to proteolytic degradation in environments such as the human body. It is therefore desirable to provide a delivery system for antibodies and other antigen-binding molecules.

### SUMMARY OF THE INVENTION

The present invention shows how to incorporate antigen-binding molecules such as antibodies into the polymeric matrix of nanospheres, while preserving their antigen-binding and biological activity. The thus encapsulated antigen-binding molecules are protected from enzymatic degradation and the surface of the nanospheres remains free for further modification such as by targeting molecules or molecules increasing the half-live of the nanospheres in the subjects body.

Thus, the invention provides a nanosphere comprising:
a) a polymeric matrix formed by one or more than one polymer comprising a main monomeric constituent selected from one or more than one of C₁-C₁₀-alkyl cyanoacrylates and C₁-C₆-alkoxy-C₁-C₁₀-alkyl cyanoacrylates; and
b) one or more than one antigen-binding molecule comprising at least one immunoglobulin light chain variable domain and at least one immunoglobulin heavy chain variable domain,
   wherein the one or more than one antigen-binding molecule is esterase-releasably incorporated in the polymeric matrix.

The invention further provides a plurality of nanospheres as described herein having a polydispersity of 0.5 or less and an average diameter of 300 nm or less as determined by Photon Correlation Spectroscopy.

The invention also provides a method for preparing nanospheres, the method comprising:
i) providing a hydrophobic liquid phase comprising one or more than one polymerizable monomer selected from C₁-C₁₀-alkyl cyanoacrylates and C₁-C₆-alkoxy-C₁-C₁₀-alkyl cyanoacrylates;
ii) finely dispersing the hydrophobic liquid phase in a hydrophilic liquid phase so as to form an emulsion, the pH of the emulsion being 4.0 or less;
iii) increasing the pH of the emulsion to a value in the range of 4.0-6.0 so as to accelerate the polymerization of the polymerizable monomer(s);
iv) then, adding one or more than one antigen-binding molecule comprising at least one immunoglobulin light chain variable domain and at least one immunoglobulin heavy chain variable domain; and
v) finally, allowing the polymerization to continue by further increasing the pH to a value not exceeding pH 8.0;
   thereby forming a suspension of nanospheres, wherein the one or more than one antigen-binding molecule is incorporated in a polymeric matrix formed by the polymerization of the polymerizable monomer(s).

The invention also provides a pharmaceutical composition comprising a plurality of nanospheres as described herein and a pharmaceutically acceptable carrier.

### BRIEF DESCRIPTION OF THE FIGURES

FIG. 1A shows the average particles sizes (Z-average diameters, columns) and polydispersities (PDI, dots) of suspensions of PBCA and PECA nanospheres prepared as described in example 1. Measurements were performed using a Zetasizer device. Transmission Electron Microscopy (TEM) images of the suspensions are shown in FIG. 1B.

FIG. 2 shows the BMP (Bone Morphogenic Protein) signaling as luminescence values measured in a luciferase reporter gene assay in the presence of different dilutions of non-purified, anti-RGMa mab loaded, esterase-treated nanospheres (“Free+encapsulated”), purified, anti-RGMa mab loaded, esterase-treated nanospheres (“encapsulated”), esterase-treated nanoparticles without anti-RGMa mab (“Empty NP”) and esterase only (“Esterase”) as described in example 4.

FIG. 3 shows the mean luminescence values and corresponding standard deviations of nanosphere samples which were calculated from the luminescence values of dilutions 4-6 depicted in FIG. 2. The mean luminescence measured for “empty NP” was normalized to 100%.

FIG. 4 shows the average particles sizes (determined as z-average diameter) and polydispersity value (PDI) of PBCA-goat IgG nanoparticle suspensions prepared as described in example 6. Sizes and PDI values were determined using a Zetasizer device.

### DETAILED DESCRIPTION OF THE INVENTION

Nanospheres are solid submicron particles having a diameter within the nanometer range (i.e. between several nanometers to several hundred nanometers) comprising a polymeric matrix, wherein further components, such as cargo molecules (e.g. antigen-binding molecules) can be incorporated (e.g. dissolved or dispersed). The nanosphere of the invention may have a size of 300 nm or less and in particular 200 nm or less, such as in the range of from 20-300 nm or, preferably, in the range of from 50-200 nm.

Unless indicated otherwise, the terms “size” and “diameter”, when referring to a basically round object such as a nanoparticle (e.g. nanospheres or nanocapsules) or a droplet of liquid, are used interchangeably.

Size and polydispersity index (PDI) of a nanoparticle preparation can be determined, for example, by Photon Correlation Spectroscopy (PCS) and cumulant analysis according to the International Standard on Dynamic Light Scattering ISO13321 (1996) and ISO22412 (2008) which yields an average diameter (z-average diameter) and an estimate of the width of the distribution (PDI), e.g. using a Zetasizer device (Malvern Instruments, Germany; software version “Nano ZS”).

The term “about” is understood by persons of ordinary skill in the art in the context in which it is used herein. In particular, “about” is meant to refer to variations of ±20%, ±10%, preferably ±5%, more preferably ±1%, and still more preferably ±0.1%.

The polymeric matrix of the nanospheres of the invention is formed by one or more than one polymer. The main monomeric constituent of the matrix-forming polymer(s) is selected from one or more than one of C₁-C₁₀-alkyl cyanoacrylates, such as C₁-C₈-alkyl cyanoacrylates, and C₁-C₆-alkoxy-C₁-C₁₀-alkyl cyanoacrylates, such as C₁-C₃-alkoxy-C₁-C₃-alkyl cyanoacrylates. For example, the main monomeric constituent of the shell-forming polymers is selected from one or more than one of methyl 2-cyanoacrylate, 2-methoxyethyl 2-cyanoacrylate, ethyl 2-cyanoacrylate, n-butyl 2-cyanoacrylate, 2-octyl 2-cyanoacrylate and isobutyl 2-cyanoacrylate, preferably from ethyl 2-cyanoacrylate and n-butyl 2-cyanoacrylate.

The term “polymeric matrix”, as used herein, describes a three-dimensional solid that is formed by one or more than one polymer. Further ingredients such as, for example, small molecule drugs and large molecule drugs such as polypeptides, e.g. antibodies and antigen-binding fragments, di- and multimers or conjugates thereof, can be incorporated, such as dissolved or dispersed, in such polymeric matrix.

The term “main monomeric constituent”, as used herein for characterizing a polymer, designates a monomeric constituent that makes up at least 80 wt-%, at least 90 wt-%, at least 95 wt-%, at least 98 wt-%, preferably at least 99 wt-% and up to 100 wt-% of the polymer.

Suitable polymers forming the matrix of the nanospheres of the invention include, but are not limited to, poly(methyl 2-cyanoacrylates), poly(2-methoxyethyl 2-cyanoacrylates), poly(ethyl 2-cyanoacrylates), poly(n-butyl 2-cyanoacrylate), poly(2-octyl 2-cyanoacrylate), poly(isobutyl 2-cyanoacrylates) and mixtures thereof, with poly(n-butyl 2-cyanoacrylates), poly(ethyl 2-cyanoacrylates) and mixtures thereof being preferred.

The weight average molecular weight of the matrix-forming polymers is typically in the range of from 1,000 to 10,000,000 g/mol, e.g. from 5,000 to 5,000,000 g/mol or from 10,000 to 1,000,000 g/mol.

The nanospheres of the invention are suitable for the delivery of antigen-binding molecules. The nanospheres of the invention protect the antigen-binding molecules on the way to the target site (e.g. the target cell) from degradation and/or modification by proteolytic and other enzymes and thus from the loss of their biological (e.g. pharmaceutical) activity. The invention is therefore also particularly useful for encapsulating antigen-binding molecules which are susceptible to such enzymatic degradation and/or modification, especially if administered by the oral route.

The term “antigen-binding molecules”, as used herein, refers to antibodies, antigen-binding fragments thereof, molecules comprising at least one antigen-binding region of an antibody as well as to antibody mimetics. The antigen-binding molecules typically have molecular weights of at least 20 kDa, in particular at least 40 kDa, for example, from 20-350 kDa or from 40-310 kDa. Preferably, an antigen-binding molecule as used in the nanospheres of the invention comprises at least one immunoglobulin domain or domain with an immunoglobulin-like fold.

The antigen-binding molecules comprised by the nanospheres of the invention can be polyclonal or monoclonal antibodies, with monoclonal antibodies being preferred. The antibodies may be naturally occurring antibodies or genetically engineered variants thereof. The antibodies may be selected from avian (e.g. chicken) antibodies and mammalian antibodies (e.g. human, murine, rat or cynomolgus antibodies), with human antibodies being preferred. The antibodies can be chimeric such as, for example, chimeric antibodies derived from murine antibodies by exchange of part or all of the non-antigen-binding regions by the corresponding human antibody regions. Where the antibody is a mammalian antibody, it may belong to one of several major classes including IgA, IgD, IgE, IgG, IgM and heavy chain antibodies (as found in camelids). IgGs (gammaglobulins) are the preferred class if mammalian antibodies because they are the most common antibodies in mammals, are specifically recognized by Fc gamma receptors and can generally be easily prepared in vitro. Where the antibody is an IgG, it may belong to one of several isotypes including IgG1, IgG2, IgG3 and IgG4. The antibodies can be prepared, for example, via immunization of animals, via hybridoma technology or recombinantly.

The antigen-binding molecules comprised by the nanospheres of the invention can be antigen-binding fragments of antibodies such as, for example, Fab, F(ab)₂ and Fv fragments.

The antigen-binding molecules comprised by the nanospheres of the invention can be molecules having at least one antigen-binding region of an antibody which can be selected from, but are not limited to, dimers and multimers of antibodies; bispecific antibodies; single chain Fv fragments (scFv) and disulfide-coupled Fv fragments (dsFv).

The antigen-binding molecules comprised by the nanospheres of the invention can also be antibody mimics. The term “antibody mimics”, as used herein, refers to artificial polypeptides or proteins which are capable of binding specifically to an antigen but are not structurally related to antibodies. For example such polypeptides and proteins may be based on scaffolds such as the Z domain of protein A (i.e. affibodies), gamma-B crystalline (i.e. affilins), ubiquitin (i.e. affitins), lipcalins (i.e. anticalins), domains of membrane receptors (i.e. avimers), ankyrin repeat motif (i.e. DARPins), the 10th type III domain of fibrection (i.e. monobodies). The term “antibody mimics” also includes dimers and multimers of such polypeptides or proteins.

The term “antigen-binding molecule” also included conjugates of an antibody or another molecule comprising at least one antigen-binding region of an antibody or an antibody mimic with, for example, at least one detectable moiety (e.g. fluorophores or enzymes) or macromolecule such as PEG or a serum protein (e.g. BSA).

The nanospheres of the invention may comprise at least 0.5 wt-%, in particular at least 5 wt-%, preferably at least 10 wt-%, and more preferably at least 15 wt-% antigen-binding molecule(s) relative to the total weight of matrix-forming polymer(s) and antigen-binding molecule(s) of the nanosphere. The amount of antigen-binding molecule(s) can be up to 10 wt-%, up to 15 wt-%, up to 20 wt-% or more relative to the total weight of matrix-forming polymer(s) and antigen-binding molecule(s).

The antigen-binding molecules are esterase-releasably incorporated in the polymeric matrix of the nanospheres of the invention. The term “esterase-releasably” means that the antigen-binding molecules can be released from the nanoparticle by the catalytic activity of an esterase. Esterases can catalyze the hydrolysis of the alkyl or alkoxyalkyl side chains of polymers, such as the matrix-forming polymers described herein, with the release of alkanol or alkoxyalkanol. It is believed that the polymer is rendered water-soluble by the action of the esterase so that the antigen-binding molecules can be leached out by aqueous liquids such as bodily fluids. “Incorporated in the polymeric matrix” means that the antigen-binding molecules may be dissolved or dispersed in the polymeric matrix.

The phrases “incorporated in the polymeric matrix of the nanosphere” and “encapsulated in the nanosphere” are used interchangeably herein. Likewise, the term “encapsulation” [of antigen-binding molecules in nanospheres of the invention] refers to the incorporation of the antigen-binding molecules in the polymeric matrix of the nanospheres. In contrast, molecules (such as antibodies) which are only attached to the surface of the nanospheres are not “encapsulated by” or “incorporated in” the polymeric matrix of the nanospheres.

Advantageously, the antigen-binding molecules encapsulated in nanospheres of the invention retain a considerable proportion of their original antigen-binding and biological activity. At least 20%, in particular at least 30%, preferably at last 40% and up to 45% or more of the antigen-binding molecules encapsulated in nanospheres of the invention may still be capable of binding to their antigen(s) after release from the nanosphere. Likewise, the antigen-binding molecules encapsulated in nanospheres of the invention may retain at least 20%, in particular at least 30%, preferably at last 40% and up to 45% or more of their original biological (e.g. pharmaceutical) activity.

The term “biological activity” refers to the effect of a compound (such as an antigen-binding molecule) on a biological system (such as a cell, a tissue or an organism). The biological activity can be determined by examining the processes affected by the biologically active compound such as, for example, the expression of particular (reporter) genes, the phosphorylation of proteins which are part of cell signaling pathways, cell viability and cell proliferation.

Methods for measuring biological activity of compounds and their binding to specific antigen(s) are well-known in the art. Examples of such methods include, but are not limited to, Enzyme-Linked Immunosorbent Assay (ELISA) and flow cytometry.

The invention further provides a plurality of nanospheres as described herein having a relatively high uniformity with respect to size. In particular, nanosphere preparations obtained with the method of the invention can have PDI (polydispersity index) values as determined by Photon Correlation Spectroscopy (PCS) of 0.5 or less, 0.3 or less, preferably 0.2 or less, or even 0.1 or less, e.g. in the range of from 0.05 to 0.5. The average diameter of the nanospheres may be 300 nm or less and in particular 200 nm or less, such as in the range of from 20-300 nm or, preferably, in the range of from 50-200 nm.

The term “plurality of nanocapsules” refers to 2 or more nanocapsules, for example at least 10, at least 100, at least 1,000, at least 5,000, at least 10,000, at least 50,000, at least 100,000, at least 500,000, or at least 1,000,000 or more nanocapsules.

Optionally, the nanospheres of the invention may further comprise one or more than one stabilizer as described herein.

The components of the nanospheres of the invention, in particular the matrix-forming polymer(s), as well as the ingredients of compositions according to the invention, in particular the carrier, are, expediently, pharmaceutically acceptable.

The term “pharmaceutically acceptable”, as used herein, refers to a compound or material that does not cause acute toxicity when nanospheres of the invention or a composition thereof is administered in the amount required for medical treatment or prophylaxis.

The nanospheres of the invention can be prepared by a modified miniemulsion polymerization method, in particular by a method comprising:
i) providing a hydrophobic liquid phase comprising one or more than one polymerizable monomer selected from C₁-C₁₀-alkyl cyanoacrylates and C₁-C₆-alkoxy-C₁-C₁₀-alkyl cyanoacrylates;
ii) finely dispersing the hydrophobic liquid phase in a hydrophilic liquid phase so as to form an emulsion, the pH of the emulsion being 4.0 or less, e.g. in the range of pH 1.0 to 3.0;
iii) increasing the pH of the emulsion to a value in the range of 4.0-6.0, in particular to a pH in the range of from 4.8-5.5 and preferably to a pH in the range of from 4.9-5.2, so as to accelerate the polymerization of the polymerizable monomer(s);
iv) then, adding one or more than one antigen-binding molecule comprising at least one immunoglobulin light chain variable domain and at least one immunoglobulin heavy chain variable domain; and
v) finally, allowing the polymerization to continue by further increasing the pH to a value not exceeding pH 8.0, in particular to a pH in the range of from 6.8-7.5 and preferably to a pH in the range of from 6.9-7.2;
   thereby forming a suspension of nanospheres, wherein the one or more than one antigen-binding molecule is incorporated in a polymeric matrix formed by the polymerization of the polymerizable monomer(s).

Without wishing to be bound by theory, it is assumed that the polymerization of the polymerizable monomer(s) comprised by the hydrophobic liquid phase of step (i) is initiated by hydroxyl ions and occurs according to the anionic polymerization mechanism (cf., e.g., Vauthier et al., Adv. Drug Deliv. Rev. 2003, 55:519-548). The polymerizable monomer(s) are selected from one or more than one of C₁-C₁₀-alkyl cyanoacrylates, such as C₁-C₈-alkyl cyanoacrylates, and C₁-C₆-alkoxy-C₁-C₁₀-alkyl cyanoacrylates, such as C₁-C₃-alkoxy-C₁-C₃-alkyl cyanoacrylates. Examples of suitable polymerizable monomer(s) include, but are not limited to, methyl 2-cyanoacrylate, 2-methoxyethyl 2-cyanoacrylate, ethyl 2-cyanoacrylate, n-butyl 2-cyanoacrylate, 2-octyl 2-cyanoacrylate, isobutyl 2-cyanoacrylate, and mixtures thereof, ethyl 2-cyanoacrylate, n-butyl 2-cyanoacrylate and mixtures thereof being preferred.

Optionally, the hydrophobic liquid phase of step (i) may further comprise one or more than one oil. The term “oil”, as used herein, refers to a neural, nonpolar substance that has a density lower than that of water, is miscible with polymerizable monomers as described herein and with other oily substances (lipophilic), is immiscible with water (hydrophobic) and is liquid at room temperature (25° C.). The oil(s) use in step (i) of the method of the invention may be of petrochemical, animal or plant origin. Examples of suitable oils include, but are not limited to, canola oil, corn oil, sunflower oil, peanut oil and, in particular, soybean oil.

The hydrophilic liquid phase used in step (ii) is typically an acidic aqueous solution, for example an aqueous solution of an inorganic acid such as phosphoric acid or hydrochloric acid.

The hydrophobic and hydrophilic liquid phases are preferably prepared at room temperature and are then kept on ice at a temperature of about 0° C. until use.

The amount of the hydrophobic liquid phase is typically in the range of from 1-40 wt-%, such as in the range of from 2-25 wt-% relative to the total weight of the hydrophilic and hydrophobic liquid phases.

The hydrophilic liquid phase or the hydrophobic liquid phase or both, and preferably the hydrophilic phase, may contain one or more than one stabilizer as described herein. The term “stabilizer”, as used herein, refers to a compound capable of stabilizing an emulsion as prepared in step (ii) of the method of the invention. The stabilizers keep the individual droplets of the hydrophobic liquid phase dispersed in the hydrophilic liquid phase apart from one another and substantially prevent agglomeration thereof. Examples of suitable stabilizers include, but are not limited to, poloxamers, e.g. poloxamer 188, poloxamer 338 and poloxamer 407; sodium n-C₁₂-C₁₆ alkyl sulfates, e.g. sodium dodecyl sulfate, sodium myristyl sulfate and sodium hexadecyl sulfate; sorbitan fatty acid esters, e.g. sorbitan monoesters of monounsaturated or saturated C₁₁-C₁₈-fatty acids such as lauric acid, palmitic acid, stearic acid and oleic acid; polyoxyethylene sorbitan fatty acid esters, e.g. polyoxyethylene sorbitan monoesters and triesters of monounsaturated or saturated C₁₁-C₁₈-fatty acids such as lauric acid, palmitic acid, stearic acid and oleic acid; poloxamines, poly(oxyethylene) ethers, poly(oxyethylene) esters, polyethylene glycols, and mixtures thereof. A mixture of stabilizers comprising at least one poloxamer, in particular poloxamer 188, and at least one sodium n-C₁₂-C₁₆ alkyl sulfate, in particular sodium dodecyl sulfate, are particularly preferred. Most preferred stabilizers have an HLB in the range of from 6 to 16.

The total amount of the stabilizer(s) is typically in the range of from 5-25 wt-% relative to the total weight of the polymerizable monomers. For example, the amount of 5-25 wt-% stabilizers may be composed of a poloxamer, such as poloxamer 188, and a sodium n-C₁₂-C₁₆ alkyl sulfate, such as sodium dodecyl sulfate, in a weight ratio of 1 part sodium n-C₁₂-C₁₆ alkyl sulfate to 2-3 parts poloxamer.

In step (ii) of the method of the invention, the hydrophobic liquid phase is finely dispersed in the hydrophilic liquid phase so as to form an emulsion of fine droplets of the hydrophobic liquid distributed throughout the hydrophilic liquid. This emulsion may be obtained, by applying shear forces, for example by thorough mixing using a static mixer, by ultrasound, by homogenization under pressure, e.g. under a pressure of at least 5,000 kPa, such as from 20,000-200,000 kPa, preferably from 50,000-100,000 kPa, or by combining any of these homogenization methods. The emulsion of the hydrophobic liquid in the hydrophilic liquid can be prepared in a two-step process, wherein the two phases are first mixed, e.g. with a static mixer (rotator/stator-type mixer), so as to obtain a pre-emulsion which, in a second step, is further homogenized ultrasonically and/or using a high pressure homogenizer so as to reduce the size of the hydrophobic liquid droplets. The shear forces may be applied for a time of from 1-10 min, in particular from 2-5 min. For example, ultrasound may be applied for 1-10 min, in particular from 2-5 min, with amplitude in the range of from 50-100%.

Step (ii) may be carried out at about 25° C. (room temperature) or, preferably, at a temperature of about 0° C. (such as on ice).

The polymerization of the polymerizable monomers is initiated upon contact with the hydrophilic liquid phase but proceeds very slowly unless in an alkaline environment. In step (iii) of the method of the invention, the polymerization in the emulsion is therefore accelerated by increasing the pH of the emulsion to a value in the range of 4.0-6.0. This may be achieved by adding a base or an aqueous solution thereof. Examples of suitable bases include, but are not limited to, sodium hydroxide, potassium carbonate, ammonia and Tris (base).

After increasing the pH of the emulsion to 4.0-6.0, one or more than one antigen-binding molecule, as described herein, e.g. in the form of an aqueous solution, is added to emulsion. Thus, the antigen-binding molecules can be incorporated in the polymeric matrix of the forming nanospheres. The amount of antigen-binding molecules added in step (iv) of the method is typically in the range of from 0.05 wt-% to 20 wt-%, in particular from 0.5 wt-% to 15 wt-%, relative to the total weight of matrix-forming polymer(s) and antigen-binding molecule(s). Optionally, the mixture of antigen-binding molecule(s) and emulsion is incubated for 5-20 min at about 25° C. (room temperature).

The polymerization is continued, while increasing of the pH in step (v) to a pH not exceeding pH 8.0. This allows residual monomer to polymerize. The polymerization is usually completed after about 10-14 h (e.g. an overnight incubation) which may be carried out at a temperature of about 4° C.

Optionally, the method of the invention may further comprise purification steps such as filtration steps, and/or a partial or complete exchange of the suspension medium of the obtained nanospheres, e.g. by dialysis.

The method of the invention can yield preparations of nanospheres as described herein. In particular, the method is suitable for preparing nanospheres comprising antigen-binding molecules which, after release from the nanospheres retain at least 20%, in particular at least 30%, preferably at last 40% and up to 45% or more of their antigen-binding and original biological activity, respectively.

The method of the invention allows for a high encapsulation efficiency of the antigen-binding molecule(s). The term “encapsulation efficiency” refers to the amount of antigen-binding molecule(s) encapsulated in nanospheres relative to the total amount of antigen-binding molecule(s) used for preparing the nanospheres. Specifically, the method of the invention allows for encapsulation efficiencies of at least 50%, in particular at least 70%, at least 80%, preferably at least 90 wt-%, at least 95% or even of 99% or more.

The invention further provides a pharmaceutical composition comprising a plurality of nanospheres as described herein, and a pharmaceutically acceptable carrier. The carrier is chosen to be suitable for the intended way of administration which can be, for example, oral or parenteral administration, intravascular, subcutaneous or, most commonly, intravenous injection, transdermal application, or topical applications such as onto the skin, nasal or buccal mucosa or the conjunctiva.

The nanospheres of the invention can increase the bioavailability and efficacy of the encapsulated active agent(s) by protecting said agent(s) from premature degradation in the gastrointestinal tract and the blood, and allowing for a sustained release thereof. Following oral administration, the nanospheres of the invention can traverse the intestinal wall and even barriers such as the blood-brain barrier.

Liquid pharmaceutical compositions of the invention typically comprise a carrier selected from aqueous solutions which may comprise one or more than one water-soluble salt and/or one or more than one water-soluble polymer. If the composition is to be administered by injection, the carrier is typically an isotonic aqueous solution (e.g. a solution containing 150 mM NaCl, 5 wt-% dextrose or both). Such carrier also typically has an appropriate (physiological) pH in the range of from about 7.3-7.4.

Solid or semisolid carriers, e.g. for compositions to be administered orally or as an depot implant, may be selected from pharmaceutically acceptable polymers including, but not limited to, homopolymers and copolymers of N-vinyl lactams (especially homopolymers and copolymers of N-vinyl pyrrolidone, e.g. polyvinylpyrrolidone, copolymers of N-vinyl pyrrolidone and vinyl acetate or vinyl propionate), cellulose esters and cellulose ethers (in particular methylcellulose and ethylcellulose, hydroxyalkylcelluloses, in particular hydroxypropylcellulose, hydroxylalkylalkylcelluloses, in particular hydroxyl-propylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate), high molecular weight polyalkylene oxides (such as polyethylene oxide and polypropylene oxide and copolymers of ethylene oxide and propylene oxide), polyvinyl alcohol-polyethylene glycol-graft copolymers, polyacrylates and polymethacrylates (such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates)), polyacrylamides, vinyl acetate polymers (such as copolymers of vinyl acetate and crotonic acid, partially hydrolyzed polyvinyl acetate), polyvinyl alcohol, oligo- and polysaccharides such as carrageenans, galactomannans and xanthan gum, or mixtures of one or more thereof. Solid carrier ingredients may be dissolved or suspended in a liquid suspension of nanospheres of the invention and the liquid suspension medium may be, at least partially, removed.

### Examples

### Determination of Particle Size and Polydispersity Index

In the examples described herein, size and polydispersity index (PDI) of the prepared nanoparticles were determined by cumulant analysis as defined in the International Standard on Dynamic Light Scattering ISO13321 (1996) and ISO22412 (2008) using a Zetasizer device (Malvern Instruments, Germany) which yields a mean particle size (z-average diameter) and an estimate of the width of the distribution (PDI). The PDI, as indicated in the examples, is a dimensionless measure of the broadness of the size distribution which, in the Zetasizer software ranges from 0 to 1. PDI values of <0.05 indicate monodisperse samples (i.e. samples with a very uniform particle size distribution), while higher PDI values indicate more polydisperse samples.

### Example 1 Preparation of Polymeric Nanoparticles Loaded with Anti-Biotin Goat IgG

IgG-loaded poly(n-butyl 2-cyanoacrylate) (PBCA) nanospheres were prepared as follows:

250 μl n-butyl 2-cyanoacrylate (monomer) were mixed with 21.5 μl soybean oil so as to obtain an oil phase. 16.25 mg poloxamer 188 and 6.5 mg sodium dodecyl sulfate (SDS) were mixed with 1.3 ml 0.1 M phosphoric acid so as to obtain an aqueous phase. Both phases were kept on ice. The phases were mixed and the mixture was homogenized using a probe sonicator (Hielscher Ultrasonics GmbH, Germany, 70% amplitude, 1 cycle) for two minutes while still cooling on ice. 0.1 N sodium hydroxide (NaOH) was added dropwise to the obtained emulsion while stirring (700 rpm). As soon as the pH of the emulsion reached 5.0, 1 mg anti-biotin goat IgG was added slowly while continuing stirring. After addition of the IgG, stirring of emulsion was continued for about 10 min at room temperature. Then, the pH was increased to 7.0 by dropwise addition of 0.1 N NaOH and the sample was incubated overnight at 4° C. to allow residual monomer to polymerize.

The same procedure was repeated using ethyl 2-cyanoacrylate instead of n-butyl 2-cyanoacrylate so as to obtain IgG-loaded poly(ethyl 2-cyanoacrylate) (PECA) nanospheres.

After the overnight incubation, the obtained nanospheres suspensions were analyzed using a Zetasizer device and software as described above, filtered through a 200 nm membrane and analyzed again. The results of these analyses, i.e. size (determined as z-average diameter) and PDI of the IgG-loaded PBCA nanospheres (PBCA NP) and

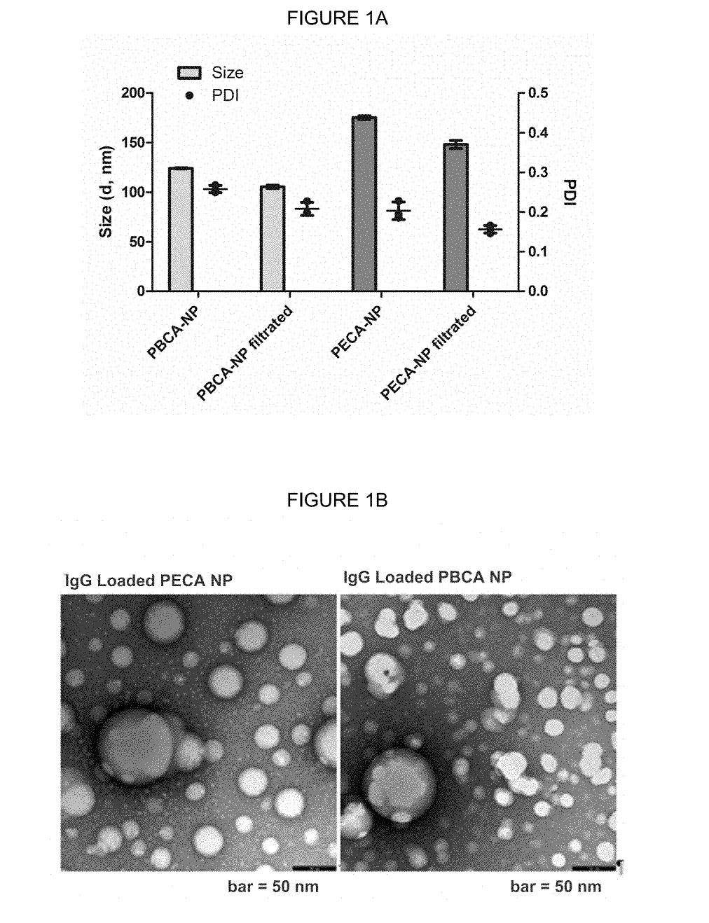

IgG-loaded PECA nanospheres (PECA NP) including standard deviations (n=3), are summarized in FIG. 1A. Additionally, the nanospheres were examined by Transmission Electron Microscopy (TEM, cf. FIG. 1B).

### Example 2 Encapsulation Efficiency (EE)

The amount of free (non-encapsulated) anti-biotin goat IgG in the PBCA nanospheres suspension of EXAMPLE 1 was determined using size exclusion high performance liquid chromatography (SE-HPLC). Only 5.6% IgG were found to be free (i.e. dissolved in suspension medium rather than encapsulated in nanospheres). The encapsulation efficiency, calculated as the quotient of [(total amount of IgG added)-(non-encapsulated IgG)]/[total amount of IgG added], was 94.4%.

### Example 3 Antigen-Binding Activity of Encapsulated IgG

250 μl n-butyl 2-cyanoacrylate (monomer) were mixed with 21.5 μl soybean oil so as to obtain an oil phase. 16.25 mg poloxamer 188 and 6.5 mg sodium dodecyl sulfate (SDS) were mixed with 1.3 ml 0.1 M phosphoric acid so as to obtain an aqueous phase. Both phases were kept on ice. The phases were mixed and the mixture was homogenized using a probe sonicator (Hielscher Ultrasonics GmbH, Germany, 100% amplitude, 1 cycle) for five minutes while still cooling on ice so as to obtain an emulsion. 500 μl of the emulsion was diluted with 800 μl aqueous phase having a composition as indicated above. 0.1 N sodium hydroxide (NaOH) was added dropwise while stirring (300-500 rpm). As soon as the pH of the emulsion reached 5, 1 mg non-specific goat IgG (without specific binding activity to biotin) or 1 mg anti-biotin goat IgG (binding specifically to biotin) was added slowly while continuing stirring. After addition of the IgG, the pH was increased to 7 by dropwise addition of 0.1 N NaOH and the sample was incubated overnight at 4° C. to allow residual monomer to polymerize.

Part of each sample (final concentration: 1.08 mg/ml PBCA) was treated with porcine liver esterase (Sigma Aldrich Co., Germany, cat. no. E2884, ≧150 U/ml, final concentration: 0.5 mg/ml) for 4 h at 37° C. while shaking.

The biotin binding activity of the samples was determined ELISA on biotin-coated microtiter plates. 6 different dilutions (serial 1:2 dilutions) were measured for each of the samples. The theoretical concentrations of anti-biotin antibodies were calculated as if all anti-biotin IgG retained antigen-binding activity. The actual concentrations of antigen-binding anti-biotin IgG were determined via ELISA (detecting with an anti-goat antibody horseradish peroxidase conjugate and tetramethylbenzidine) on the basis of an anti-biotin IgG calibrator curve covering the range of from 3.9-1,000 ng/ml anti-biotin IgG. The percentages of ELISA-detectable, antigen-binding anti-biotin IgG relative to the theoretical concentrations were calculated. The results are summarized in Table 1.

TABLE 1
Concentrations of functional anti-biotin antibodies

| 200 U esterase | | 15 U esterase | |
|---|---|---|---|
| goat IgG | anti-biotin | goat IgG | anti-biotin |
| (control) | goat IgG | (control) | goat IgG |

Theoretical concentrations [ng/ml]

| | | | | |
|---|---|---|---|---|
| dilution 1 | 318.0 | 254.0 | 414.0 | 338.0 |
| dilution 2 | 159.0 | 127.0 | 207.0 | 169.0 |
| dilution 3 | 79.5 | 63.5 | 103.5 | 84.5 |
| dilution 4 | 39.8 | 31.8 | 51.8 | 42.3 |
| dilution 5 | 19.9 | 15.9 | 25.9 | 21.1 |
| dilution 6 | 9.9 | 7.9 | 12.9 | 10.6 |

Concentrations as measured via ELISA [ng/ml]

| | | | | |
|---|---|---|---|---|
| dilution 1 | 2.8 | 136.1 | 11.1 | 184.5 |
| dilution 2 | 2.1 | 61.2 | 7.4 | 82.2 |
| dilution 3 | 1.5 | 24.9 | 6.4 | 49.4 |
| dilution 4 | 1.2 | 17.0 | 5.4 | 22.8 |
| dilution 5 | 1.5 | 7.1 | 2.1 | 9.4 |
| dilution 6 | n.d. | 6.9 | 2.7 | 6.9 |

Measured concentrations relative to theoretical concentrations [%]

| | | | | |
|---|---|---|---|---|
| dilution 1 | 0.87 | 53.60 | 2.68 | 54.58 |
| dilution 2 | 1.33 | 48.21 | 3.56 | 48.61 |
| dilution 3 | 1.92 | 39.15 | 6.20 | 58.42 |
| dilution 4 | 3.10 | 53.62 | 10.42 | 53.94 |
| dilution 5 | 7.58 | 44.42 | 8.20 | 44.54 |
| dilution 6 | 0.00 | 86.25 | 21.08 | 65.64 |
| Mean [%] | 3.0 | 47.8 | 6.2 | 52.0 |

The non-encapsulated 5.6% anti-biotin IgG (cf. EXAMPLE 2) as well as the background signal of non-biotin specific goat IgG (control) were taken into account. Accordingly, the amount of antigen-binding IgG that was esterase-releasably encapsulated in the nanospheres was about 40-45%.

### Example 4 Biological Activity of Encapsulated IgG

The biological activity of encapsulated IgG was determined in PBCA nanospheres loaded with a monoclonal antibody (mab) against Repulsive Guidance Molecule A (RGMa) as follows:

A suspension of anti-RGMa mab-loaded PBCA nanospheres was prepared using the method described in EXAMPLE 1 (adding 2.26 mg of the mab instead of 1 mg goat IgG) and contained free and encapsulated mab (sample name after esterase treatment: “Free+encapsulated”). The nanospheres of part of the suspension were separated from free mab by ultrafiltration (Amicon Cell and Biomax 500 kDa filter membrane), thus obtaining a sample that contained only encapsulated mab (sample name after esterase treatment: “encapsulated”). Part of each sample (9.55 mg/ml PBCA, 1:10 dilution) was treated with porcine liver esterase (Sigma Aldrich Co., Germany cat. no. E2884, ≧150 U/ml, final concentration: 0.22 mg/ml) for 4 h at 37° C. while shaking to release encapsulated mab from the nanospheres. As a control, PBCA nanoparticles were prepared without loading any antibody and treated with esterase as described for samples “Free+encapsulated” and “encapsulated” (sample name: “Empty NP”).

The biological anti-RGMa mab activity in each of the samples was determined via luciferase reporter gene assay using the One-Glo Luciferase Assay System (Promega, Germany). Said assay is based on the binding of Bone Morphogenic Protein (BMP) to the BMP receptor BMPR I/II located in the cell membrane of c-293 HEK cells expressing human RGMa and comprising a luciferase reporter that is responsive to BMP induced signaling of BMPR I/II. RGMa binds to BMP-2, BMP-4 or BMP-6 and acts as a co-receptor, leading to an enhanced BMP signaling. Biologically active anti-RGMa mab prevents binding of RGMa to BMP and thus reduces BMP signaling.

A 96-well plate (Corning, white assay plate) was seeded with 50,000 c-293 HEK cells (in 50 μl medium) per well. 25 μl of a sample dilution per well was added. The compositions of the dilutions are summarized in Table 2.

TABLE 2
Composition of the sample dilutions used in the luciferase assay

| concentration after dilution [ug/ml] | | | | |
|---|---|---|---|---|
| anti-RGMa mab¹ | PBCA² | esterase | dilution factor | |
| 8.2182 | 95.4545 | 10.5480 | 10 | Dilution 1 |
| 4.1091 | 47.7273 | 5.2740 | 20 | Dilution 2 |
| 2.0545 | 23.8636 | 2.6370 | 40 | Dilution 3 |
| 1.0273 | 11.9318 | 1.3185 | 80 | Dilution 4 |
| 0.5136 | 5.9659 | 0.6593 | 160 | Dilution 5 |
| 0.2568 | 2.9830 | 0.3296 | 320 | Dilution 6 |

¹absent in dilutions of the controls “Empty NP” and “Esterase”
²calculated as PBCA equivalent as if not hydrolyzed by esterase treatment, absent in the control “Esterase”

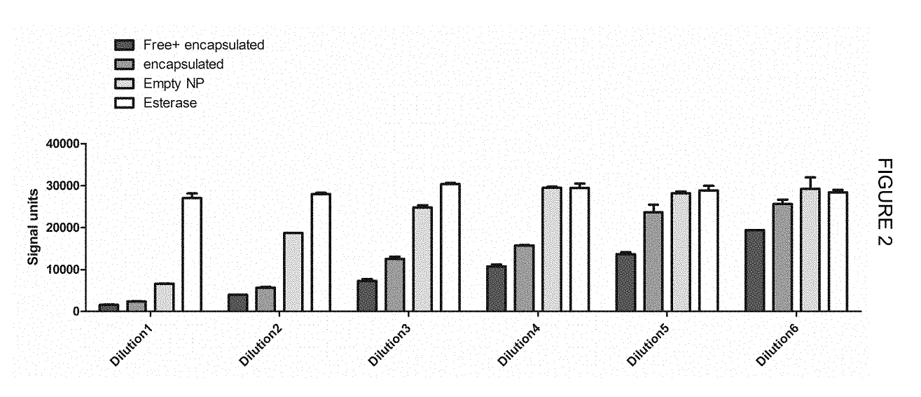

The 96-well plate was incubated for 24 h at 37° C. and 5% CO₂. Then, 75 μl/well One-Glo substrate was added. After further incubation for 7 min at room temperature while shaking at 750 rpm in the dark, the luminescence in each well was measured. The results are shown in FIG. 2.

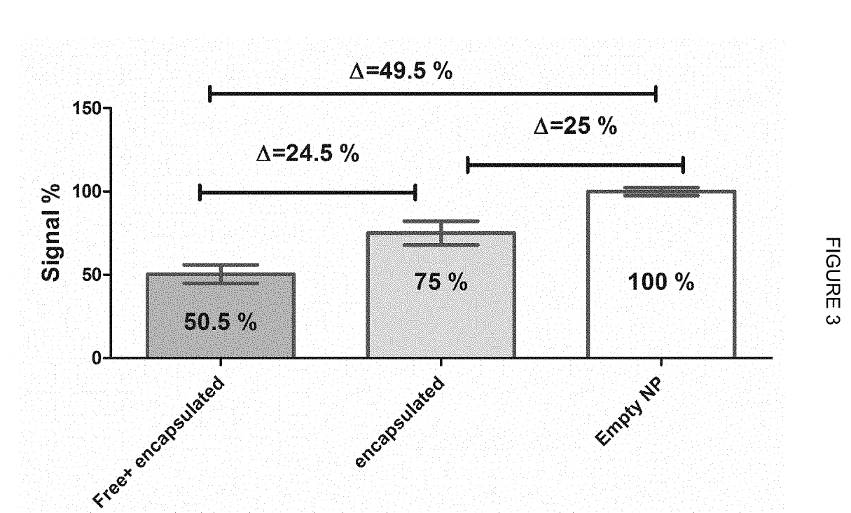

Esterase per se (sample name: “Esterase”) did not have a great effect on signal performance in all tested concentrations. However, PBCA nanoparticles without mab (“empty NP”) and its degradation products resulting from esterase treatment decreased cell signaling in Dilutions 1-3. The calculation was therefore based on the luminescence values measured for Dilutions 4-6. The mean signal value of the “empty NP” sample was normalized to 100% (cf. FIG. 3). The anti-RGMa mab from purified mab-loaded nanospheres (“encapsulated”) resulted in a 25% decrease of BMP signaling. The reduction of BMP signaling of 49.5% observed in the sample “Free+encapsulated” indicates that 24.5% of the anti-RGMa mab was free (not encapsulated in nanospheres). These results indicate that the at least 25% of the mab encapsulated in nanospheres retained its original biologically activity.

### Example 5 Preparation of PBCA Nanoparticles Loaded with Human IgG-FITC Conjugate

A suspension of PBCA nanospheres loaded with a human IgG-FITC conjugate was prepared using the method described in EXAMPLE 1, except for incubating for about 4.5 h at room temperature (instead of overnight at 4° C.) after the pH of the emulsion was adjusted to 7.0.

Prior to filtration, the z-average diameter of the nanospheres was 173 nm and the PDI 0.186. After filtration (200 nm membrane), the z-average diameter of the nanospheres was 144 nm and the PDI 0.157.

Encapsulation efficiency, determined as described in EXAMPLE 2, was 97.6% (i.e. 2.4% free antibody conjugate).

### Example 6 Preparation of PBCA Nanoparticles Loaded with Goat IgG

For each sample, 21.5 μl soybean oil was carefully mixed with the amount of n-butyl 2-cyanoacrylate (monomer) indicated in Table 3 so as to obtain an oil phase. 16.25 mg poloxamer 188 and 6.5 mg sodium dodecyl sulfate (SDS) were mixed with 1.3 ml 0.1 M phosphoric acid so as to obtain an aqueous phase. Both phases were kept on ice. The phases were mixed and the mixture was homogenized using a probe sonicator (Hielscher Ultrasonics GmbH, Germany, 1 cycle) for the time and under the conditions indicated in Table 3. 0.1 N sodium hydroxide (NaOH) was added dropwise to the obtained emulsion while stirring (300-500 rpm). As soon as the pH of the emulsion reached the value indicated in Table 3, 1 mg anti-biotin goat IgG was added slowly while continuing stirring. After addition of the IgG, stirring of emulsion was continued for about 10 min at room temperature. Then, the pH was increased to about 6.0-7.0 by dropwise addition of 0.1 N NaOH and the sample was incubated overnight at 4° C. to allow residual monomer to polymerize.

TABLE 3
Miniemulsion polymerization - conditions

| | n-butyl | | sonication | | pH when |
|---|---|---|---|---|---|
| | 2-cyano- | sonication | amplitude | sonication | adding |
| Sample | acrylate [mg] | time [min] | [%] | temperature | IgG |
| DoE1 | 100 | 2 | 100 | RT* | 7 |
| DoE2 | 100 | 2 | 50 | ice cooling | 5 |
| DoE3 | 10 | 5 | 100 | ice cooling | 3 |
| DoE4 | 10 | 5 | 100 | RT* | 5 |
| DoE5 | 10 | 5 | 50 | RT* | 3 |
| DoE7 | 100 | 5 | 100 | RT* | 3 |
| DoE8 | 10 | 5 | 50 | ice cooling | 5 |
| DoE9 | 100 | 5 | 50 | RT* | 5 |
| DoE10 | 10 | 2 | 100 | ice cooling | 5 |
| DoE11 | 10 | 2 | 50 | RT* | 5 |
| DoE12 | 100 | 2 | 50 | RT* | 3 |
| DoE13 | 100 | 5 | 50 | ice cooling | 3 |
| DoE14 | 10 | 2 | 100 | RT* | 3 |
| DoE15 | 100 | 5 | 100 | ice cooling | 5 |
| DoE16 | 100 | 2 | 100 | ice cooling | 3 |

*RT = room temperature

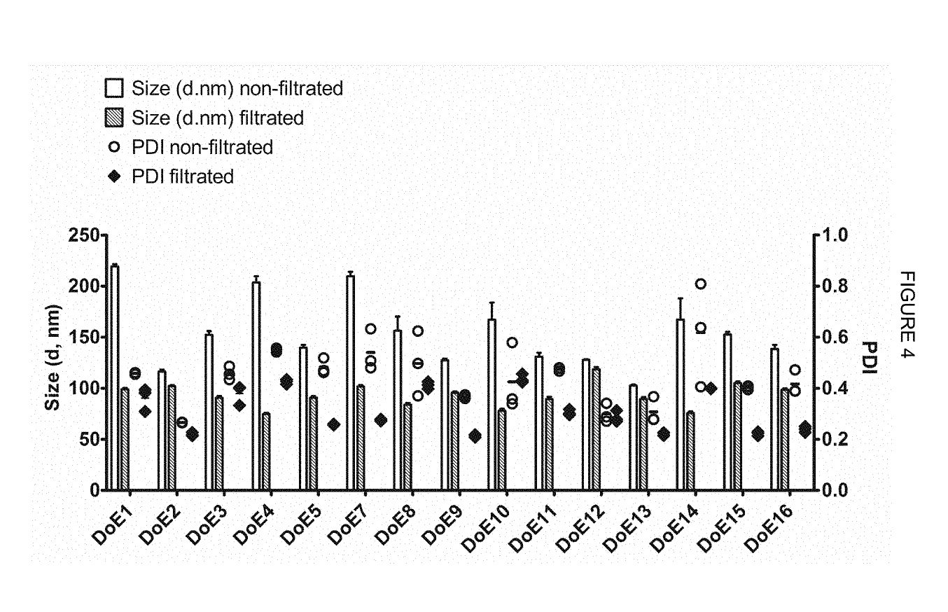

After the overnight incubation, the obtained nanospheres suspensions were analyzed using a Zetasizer device and software as described above, filtered through a 200 nm membrane and analyzed again. The results of these analyses, i.e. size (determined as z-average diameter) and PDI of the nanospheres including standard deviations (n=3), are summarized in FIG. 4. Additionally, the nanospheres were examined by Transmission Electron Microscopy (TEM).

Encapsulation efficiency (EE) of each sample was determined as described in EXAMPLE 2. The results are indicate in Table 4

TABLE 4
Encapsulation efficiency (EE)

| Sample | free IgG [%] | EE [%] |
|---|---|---|
| DoE1 | 23.29 | 76.71 |
| DoE2 | 9.22 | 90.78 |
| DoE3 | 0.29 | 99.71 |
| DoE4 | 7.62 | 92.38 |
| DoE5 | 0.29 | 99.71 |
| DoE7 | 0.61 | 99.39 |
| DoE8 | 0.56 | 99.44 |
| DoE9 | 0.29 | 99.71 |
| DoE10 | 1.47 | 98.53 |
| DoE11 | 21.87 | 78.13 |
| DoE12 | 0.29 | 99.71 |
| DoE13 | 0.29 | 99.71 |
| DoE14 | 0.29 | 99.71 |
| DoE15 | 0.29 | 99.71 |
| DoE16 | 0.29 | 99.71 |

1. A nanosphere comprising:
a) a polymeric matrix formed by one or more than one polymer comprising a main monomeric constituent selected from one or more than one of C₁-C₁₀-alkyl cyanoacrylates and C₁-C₆-alkoxy-C₁-C₁₀-alkyl cyanoacrylates; and
b) one or more than one antigen-binding molecule comprising at least one immunoglobulin light chain variable domain and at least one immunoglobulin heavy chain variable domain,
wherein the one or more than one antigen-binding molecule is esterase-releasably incorporated in the polymeric matrix.

2. The nanosphere of claim 1, wherein the one or more than one antigen-binding molecule is selected from gammaglobulins, antibody dimers, and Fab fragments and F(ab)₂ fragments.

3. The nanosphere of claim 1, wherein at least 20% of the antigen-binding molecule(s) is still capable of binding to its antigen after release from the nanosphere.

4. The nanosphere of claim 1, wherein the antigen-binding molecules released from the nanosphere retain at least 20% their original biological activity as measured with a biological assay such as a cell assay.

5. The nanosphere of claim 1, wherein the main monomeric constituent of the matrix-forming polymer(s) is selected from one or more than one of methyl 2-cyanoacrylate, 2-methoxyethyl 2-cyanoacrylate, ethyl 2-cyanoacrylate, n-butyl 2-cyanoacrylate, 2-octyl 2-cyanoacrylate and isobutyl 2-cyanoacrylate.

6. The nanosphere of claim 1, wherein the one or more than one matrix-forming polymer is selected from poly(n-butyl 2-cyanoacrylate), poly(ethyl 2-cyanoacrylate), and mixtures thereof.

7. A plurality of nanospheres of claim 1 having a polydispersity in the range of 0.5 or less as determined by cumulant analysis according to ISO13321 and ISO22412 and an average diameter in the range of 20-300 nm as determined by Photon Correlation Spectroscopy.

8. A method for preparing nanospheres, the method comprising:
i) providing a hydrophobic liquid phase comprising one or more than one polymerizable monomer selected from C₁-C₁₀-alkyl cyanoacrylates and C₁-C₆-alkoxy-C₁-C₁₀-alkyl cyanoacrylates;
ii) finely dispersing the hydrophobic liquid phase in a hydrophilic liquid phase so as to form an emulsion, the pH of the emulsion being 4.0 or less;
iii) increasing the pH of the emulsion to a value in the range of 4.0-6.0 so as to accelerate the polymerization of the polymerizable monomer(s);
iv) then, adding one or more than one antigen-binding molecule comprising at least one immunoglobulin light chain variable domain and at least one immunoglobulin heavy chain variable domain; and
v) finally, allowing the polymerization to continue by further increasing the pH to a value not exceeding pH 8.0;
thereby forming a suspension of nanospheres, wherein the one or more than one antigen-binding molecule is incorporated in a polymeric matrix formed by the polymerization of the polymerizable monomer(s).

9. The method of claim 8, wherein the nanospheres are as defined in claim 2.

10. The method of claim 8, wherein step (ii) is carried out by homogenization under pressure and/or ultrasonically.

11. The method of claim 8, wherein in step (iii) the pH is increased to a value in the range of 4.8-5.5.

12. The method of claim 8, wherein the emulsion is incubated for 5-20 min at room temperature after addition of the antigen-binding molecule(s).

13. The method of claim 8, wherein in step (v) the pH of the emulsion is increased to be in the range of 6.8-7.5.

14. The method of claim 8, wherein the amount of the hydrophobic liquid phase is from 1-40 wt-% relative to the total weight of the hydrophilic and hydrophobic liquid phases.

15. The method of claim 8, wherein the hydrophilic liquid phase or the hydrophobic liquid phase or both contain(s) one or more than one stabilizer.

16. The method of claim 15, wherein the amount of the stabilizer(s) is from 5-25 wt-% relative to the total weight of the polymerizable monomers.

17. The method of claim 15, wherein the one or more than one stabilizer is selected from poloxamers, sodium n-C₁₂-C₁₆-alkyl sulfate, sorbitan fatty acid esters, polyoxyethylene sorbitan fatty acid esters, poloxamines, poly(oxyethylene) ethers, poly(oxyethylene) esters, polyethylene glycols, and mixtures thereof.

18. The method of claim 8, wherein the one or more than one polymerizable monomer is selected from the group consisting of methyl 2-cyanoacrylate, 2-methoxyethyl 2-cyanoacrylate, ethyl 2-cyanoacrylate, n-butyl 2-cyanoacrylate, 2-octyl 2-cyanoacrylate and isobutyl 2-cyanoacrylate.

19. The method of claim 8, wherein the one or more than one antigen-binding molecule is selected from gammaglobulins, antibody dimers, and Fab fragments and F(ab)₂ fragments.

20. A nanosphere obtainable by the method of claim 8.

21. A pharmaceutical composition comprising a plurality of nanospheres according to claim 1, and a pharmacologically acceptable carrier.
